(19) United States
(12) Patent Application Publication
(10) Pub. No.: US 2003/0083257 A1
(43) Pub. Date: May 1, 2003

(54) Modified cDNA for high expression levels of factor VIII and its derivatives

(76) Inventors: Claude Negrier, Irigny (FR); Jean-Luc Plantier, Gringy (FR); Marie-Helene Rodriguez, L'isle d'Abeau (FR); Hans-Peter Hauser, Marburg (DE)

Correspondence Address: FINNEGAN, HENDERSON, FARABOW, GARRETT &, DUNNER LLP, 1300 I STREET, NW, WASHINGTON, DC, 20006, US

(21) Appl. No.: 10/210,036

(22) Filed: Aug. 2, 2002

(30) Foreign Application Priority Data
Aug. 8, 2001 (EP) 01118775.4

(51) Int. Cl. A61K 35/14; C07K 17/00; C07K 16/00; C07K 14/00; C07K 1/00; C12N 5/02; C12N 5/00; C12N 15/74; C12N 15/70; C12N 15/09; C12N 15/00; C12P 21/04; C12P 21/06; C07H 21/04; A61K 38/00

(52) U.S. Cl. 514/12; 435/69.1; 536/23.5; 435/320.1; 530/383; 435/325

(57) ABSTRACT

A modified factor VIII CDNA is disclosed, wherein one or more spliceable nucleotide sequences have been inserted into introns 1 and/or 13 of the wild-type factor VIII cDNA. Further, a process for the production of a biologically active recombinant human factor VIII or its derivative is disclosed, which is performed by cultivating an animal cell line comprising a recombinant expression vector containing said modified factor VIII cDNA. Moreover, a transfer vector for use in the human gene therapy is described which comprises said modified factor VIII cDNA. Finally, the use of recombinant human factor VII and its derivatives for the treatment of hemophilia is disclosed.

The present application claims benefit of priority of European Patent Application No. 01118775.4, filed Aug. 8, 2001, the disclosure of which is incorporated herein by reference in its entirety.

The present invention relates to modified DNA sequences coding for biologically active recombinant human factor VIII and its derivatives, recombinant expression vectors containing such DNA sequences, host cells transformed with such recombinant expression vectors, processes for the manufacture of the recombinant human factor VIII and its derivatives, and use of the recombinant human factor VII and its derivatives for the treatment of hemophilia. The invention also covers a transfer vector for use in human gene therapy, which comprises such modified DNA sequences.

Classic hemophilia, or hemophilia A, is the most common of the inherited bleeding disorders. It results from a chromosome X-linked deficiency of blood coagulation factor VIII and affects almost exclusively males with an incidence of between one and two individuals per 10,000. The X-chromosome defect is transmitted by female carriers who are not themselves hemophiliacs. The clinical manifestation of hemophilia A is an abnormal bleeding tendency. Before treatment with factor VIII concentrates was introduced, the mean life span for a person with severe hemophilia was less than 20 years. The use of concentrates of factor VIII from plasma has considerably improved the situation for the hemophilia patients. The mean life span has increased significantly, giving many hemophilia patients the possibility to live a more or less normal life. However, there have been certain problems with the plasma-derived concentrates and their use, the most serious of which has been the transmission of viruses. So far, viruses causing AIDS, hepatitis B, non A hepatitis and non B hepatitis have hit the hemophilia population seriously. Although different viral inactivation methods and new highly purified factor VIII concentrates have recently been developed, viral contamination is still a possibility. Also, the factor VIII concentrates are fairly expensive because of the limited supply of human plasma raw material.

Factor VIII derived from recombinant material is likely to solve problems, such as viral contamination, associated with the use of plasma-derived factor VIII concentrates for treatment of hemophilia A. However, the development of a recombinant factor VIII has met with some difficulties. There has been difficulty, for instance, achieving production levels in sufficiently high yields, while retaining the biological activity of the full-length protein.

In fresh plasma prepared in the presence of protease inhibitors, factor VIII has been shown to have a molecular weight of 280 kDa and to be composed of two polypeptide chains of 200 kDa and 80 kDa (Andersson, L.-O., et al. (1986) Proc. Natl. Aca. Sci. USA 83, 2979-2983). These chains are held together by metal ion bridges. Proteolytically degraded forms of the factor VIII molecule can be found as active fragments in factor VIII material purified from commercial concentrates (Andersson, L.-O., et al. ibid.; Andersson, L.-O., et al. (1985) EP 0 197 901). The fragmented form of factor VIII having molecular weights from 260 kDa down to 170 kDa, consists of one heavy chain with a molecular weight ranging from 180 kDa down to 90 kDa, where all variants have identical amino termini, in combination with one 80 kDa light chain. The amino-terminal region of the heavy chain is identical to that of the single chain factor VIII polypeptide that can be deduced from the nucleotide sequence data of the factor VIII cDNA (Wood, W. I., et al. (1984) Nature 312, 330-336; Vehar, G. A., et al. (1984) Nature 312, 337-342).

The smallest active form of factor VIII with a molecular weight of 170 kDa, consisting of one 90 kDa and one 80 kDa chain, can be activated with thrombin to the same extent as the higher molecular weight forms, and thus represents an unactivated form. It has also been shown to have full biological activity in vivo as tested in hemophilia dogs (Brinkhous, K. M., et al. (1985) Proc.Natl.Acad.Scl. USA 82, 8752-8756). Thus, the haemostatic effectiveness of the 170 kDa form is similar to the high molecular weight forms of factor VIII.

The fact that the heavily glycosylated region of the factor VIII polypeptide chain, residing between amino acids Arg-740 and Glu-1649, does not seem to be necessary for full biological activity has prompted several researchers to attempt to produce derivatives of recombinant factor VIII lacking this region. This has been achieved by deleting a portion of the cDNA encoding the heavily glycosylated region of factor VIII either entirely or partially.

For example, J. J. Toole, et al. reported the construction and expression of factor VIII lacking amino acids 982 through 1562, and 760 through 1639 (Proc.Natl. Acad.Sci. USA (1986) 83, 5939-5942). D. L. Eaton, et al. reported the construction and expression of factor VIII lacking amino acids 797 through 1562 (Biochemistry (1986) 25, 8343-8347). R. J. Kaufman described the expression of factor VIII lacking amino acids 741 through 1646 (PCT application No. WO 87/04187). N. Sarver, et al. reported the construction and expression of factor VIII lacking amino acids 747 through 1560 (DNA (1987) 6, 553-564). M. Pasek reported the construction and expression of factor VIII lacking amino acids 745 through 1562, and amino acids 741 through 1648 (PCT application No. WO 88/00831). K.-D. Langner reported the construction and expression of factor VIII lacking amino acids 816 through 1598, and amino acids 741 through 1689 (Behring Inst. Mitt., (1988) No. 82, 16-25, EP 295 597). P. Meulien, et al. reported the construction and expression of factor VIII lacking amino acids 868 through 1562, and amino acids 771 through 1666 (Protein Engineering (1988) 2(4), 301-306, EP 0 303 540 A1). When expressing these deleted forms of factor VIII cDNA in mammalian cells the production level is typically 10 times higher as compared to full-length factor VIII.

Furthermore, attempts have been made to express the 90 kDa and 80 kDa chains separately from two different cDNA derivatives in the same cell (Burke, R. L., et al. (1986), J. Biol. Chem. 261, 12574-12578, Pavirani, A., et al. (1987) Biochem. Biophys. Res. Comm., 145, 234-240). However, in this system the in vivo reconstitution seems to be of limited efficiency in terms of recovered factor VIII; C activity.

Several studies have described the mechanisms by which the production of FVIII may be hindered. First, within the FVIII cDNA sequence two nucleotides stretches, localized in the A2 coding domain, were demonstrated to act as transcriptional silencers (Fallaux et al., 1996; Hoeben et al., 1995; Koeberl et al., 1995; Lynch et al., 1993): Second, FVIII protein synthesis is tightly regulated by several reticulum endoplasmic chaperones (BIP; Calreticulin; Calnexin; ERGIC-53). Interactions with these chaperones may retain FVIII in the cell and direct it through the cellular degradation machinery (Dorner et al., 1987; Nochols et al., 1998; Pipe et al., 1998). Third, FVIII, once secreted, is sensitive to protease degradation unless it is protected by molecules such as the von Willebrand Factor (vWF) (Kaufman et al., 1989).

It is therefore difficult to develop processes that result in higher yields of FVIII. The present invention describes modified, recombinant FVIII cDNA that is useful to produce enhanced yields of biologically active FVIII, which can be used in pharmaceutical preparations.

### BRIEF DESCRIPTION OF THE DRAWINGS

FIG. 1 shows insertion of FIX intron in the FVIII cDNA.

FIG. 2 shows the APOAI intron fragment (SEQ ID NO:5) and BGLOBI intron fragment (SEQ ID NO:6).

FIG. 3 illustrates the pCR2.1-ABC and pCR2.1-ABC13 plasmids.

FIG. 4 shows insertion of APOAI intron 1 in the first position.

FIG. 5 illustrates the plasmids obtained after the first cloning step.

FIG. 6 shows the pCR2.1 insertion of the introns in the full-length FVIII-ΔB cDNA.

FIG. 7 shows pcDNA3-FVIII A1+13 and pcDNA3-FVIII B1+13 plasmids.

As used herein, “spliceable” refers to the ability of a length of DNA sequence to be excised from the DNA sequence it was joined with, followed by a rejoining of the DNA from which the fragment was excised. Splicing generally occurs in the nucleus before transport into the cytoplasm. One of ordinary skill in the art would be able to determine if a length of sequence was indeed spliced from the sequence it was joined with.

“Modified factor VIII cDNA” as used in the application refers to wild-type factor VIII cDNA to which deletions, substitutions, or additions of DNA molecules, have been made. Proteins encoded by these cDNAs retain biological activity similar to the protein encoded by wild-type factor VIII cDNA.

Similarly, “derivative” refers to a protein encoded by a modified factor VIII cDNA. Thus, derivative refers to a wild-type factor VIII protein to which deletions, substitutions, or additions of amino acids have been made. These molecules retain biological activity similar to wild-type factor VIII protein.

As used herein, “wild-type” refers to the naturally occurring form of a DNA, cDNA, or protein molecule. Naturally occurring includes allelic variations that may exist between individuals, various degrees of glycosylation, and different post-translational modifications that may naturally occur.

“Synthetic intron” refers to a naturally occurring segment of genomic DNA that is transcribed, but removed from within the transcript by splicing together the sequences (exons) on either side of it.

This invention relates to, among many embodiments, modified factor VIII cDNA molecules that code for recombinant factor VIII derivatives, corresponding, with regard to molecular weight and other biochemical characteristics, to a previously derived plasma factor VIII form present in considerable amounts in commercial concentrates (Andersson, L.-O. et al., (1986), Proc. Natl. Acad. Sci. USA 83, 2979-2983). These new factor VIII cDNA derivatives give sufficiently high yields of recombinant factor VIII protein to be used in an industrial process for a pharmaceutical preparation of recombinant factor VIII or its derivatives.

This invention is based on the observation that the lack of introns 1 and/or 13 of the wild-type factor VIII genomic DNA in the cDNA of factor VIII prevents high level expression of factor VIII. It could therefore be hypothesized that addition of these introns to the cDNA, or a similar modification, would lead to high level expression of factor VIII. This can effectively be done by the insertion of a truncated FIX intron in the position of intron 1 and/or 13 as described in European patent application 1 048 726. However, it was unexpectedly found that this effect is not restricted to a truncated FIX intron and that the introduction of one or more spliceable nucleotide sequences into the position of introns 1 and/or 13 of the wild-type factor VIII genomic DNA increases the level of expression of factor VIII considerably.

Thus, one embodiment of the invention is a modified factor VIII cDNA, comprising at least one spliceable nucleotide sequence that is inserted into at least one intron of the wild-type factor VIII cDNA. In additional embodiments, a spliceable nucleotide sequence is inserted into the position of intron 1, intron 13, or both intron 1 and intron 13 of the wild-type factor VIII genomic DNA. In yet another embodiment, the spliceable nucleotide sequence may comprise a full-length or fragment of an intron that is part of another gene's genomic sequence. Specific embodiments include insertion of the first complete or truncated Apolipoprotein A1 intron or the first complete or truncated β-Globin intron into the position of intron 1, intron 13, or both intron 1 and intron 13 of the wild-type factor VIII genomic DNA.

A further embodiment of this invention is to improve the level of expression of factor VIII and its derivatives by use of a modified factor VIII cDNA in which the B-domain of the wild-type factor VIII cDNA has been shortened or completely eliminated.

In yet another embodiment, a modified factor VIII cDNA is used that comprises a first DNA segment coding for the amino acids 1 through 740 of the human factor VIII and a second DNA segment coding for the amino acids 1649 through 2332 of the human factor VIII. These two segments may be interconnected by a linker DNA segment preferably coding for a linker peptide of at least two amino acids which are selected from lysine or arginine as described in International patent application WO 92/16557.

In one embodiment of the invention, such B-domain deleted factor VIII cDNA may comprise at least one spliceable nucleotide sequence that is inserted into the position of at least one intron of the wild-type factor VIII genomic DNA. In yet another embodiment, a spliceable nucleotide sequence is inserted at the position of intron 1, intron 13, or both intron 1 and intron 13 of the wild-type factor VIII genomic DNA.

In additional embodiments, the spliceable nucleotide sequence may comprise a full-length or fragment of a synthetic intron. Specific embodiments include insertion of the first complete or truncated Apolipoprotein A1 intron or the first complete or truncated β-Globin intron into the position of intron 1, intron 13, or both intron 1 and intron 13 of the wild-type factor VIII genomic DNA.

The production of factor VIII proteins at high levels in suitable host cells requires the assembly of the above-mentioned modified factor VIII cDNA's into efficient transcriptional units together with suitable regulatory elements in a recombinant expression vector. These recombinant vectors can then be propagated, for instance in E. coli, according to methods known to those skilled in the art. Efficient transcriptional regulatory elements could be derived from viruses having animal cells as their natural hosts or from the chromosomal DNA of animal cells. Preferably, promoter-enhancer combinations derived from the Simian Virus 40, adenovirus, BK polyoma virus, human cytomegalovirus, or the long terminal repeat of Rous sarcoma virus, or promoter-enhancer combinations including strongly constitutively transcribed genes in animal cells like beta-actin or GRP78 are used. In order to achieve stable, high levels of mRNA transcribed from the factor VIII cDNA's, the transcriptional unit should contain in its 3′-proximal part a DNA region encoding a transcriptional termination-polyadenylation sequence. Preferably, this sequence is derived from the Simian Virus 40 early transcriptional region, the rabbit beta-globin gene, or the human tissue plasminogen activator gene.

The factor VIII cDNA's thus assembled into efficient recombinant expression vector are then introduced into a suitable host cell line for expression of the factor VIII proteins. Preferably this cell line is an animal cell line of vertebrate origin in order to ensure correct folding, disulfide bond formation, asparagine-linked glycosylation and other post-translational modifications, as well as secretion into the culture medium. Examples of other post-translational modifications include tyrosine O-sulfation and proteolytic processing of the nascent polypeptide chain.

Examples of cell lines that can be used are monkey COS-cells, mouse L-cells, mouse C127-cells, hamster BHK-21 cells, human embryonic kidney 293 cells, and preferentially CHO-cells.

The recombinant expression vector encoding the factor VIII cDNA's can be introduced into an animal cell line in several different ways. For instance, recombinant expression vectors can be created from vectors based on different animal viruses. In an embodiment of the invention, the recombinant expression vector of the invention is introduced into an animal cell line via vectors based on baculovirus, vaccinia virus, adenovirus, and preferably bovine papilloma virus.

The transcription units encoding the factor VIII cDNA's can also be introduced into animal cells together with another recombinant gene. Specifically, the additional recombinant gene may function as a dominant selectable marker in these cells in order to facilitate the isolation of specific cell clones that have integrated the recombinant DNA into their genome. Examples of this type of dominant selectable marker genes are Tn5 aminoglycoside phosphotransferase, conferring resistance to Geneticin (G418), hygromycin phosphotransferase, conferring resistance to hygromycin, and puromycin acetyl transferase, conferring resistance to puromycin. The recombinant expression vector encoding such a selectable marker can reside either on the same vector as the one encoding the factor VIII cDNA, or it can be encoded on a separate vector which is simultaneously introduced and integrated to the genome of the host cell, frequently resulting in a tight physical linkage between the different transcription units.

Other types of selectable marker genes that can be used together with the factor VIII cDNA's are based on various transcription units encoding dihydrofolate reductase (dhfr). After introduction of this type of gene into cells lacking endogenous dhfr-activity, preferentially CHO-cells (DUKX-B11, DG-44), it will enable these to grow in media lacking nucleosides. An example of such a medium is Ham's F12 without hypoxanthin, thymidin, and glycine. These dhfr-genes can be introduced together with the factor VIII cDNA transcriptional units into CHO-cells of the above type, either linked on the same vector or on different vectors, thus creating dhfr-positive cell lines producing recombinant factor VIII protein.

If the above cell lines are grown in the presence of the cytotoxic dhfr-inhibitor methotrexate, new cell lines resistant to methotrexate will emerge. These cell lines may produce recombinant factor VIII protein at an increased rate due to the amplified number of linked dhfr and factor VIII transcriptional units. When propagating these cell lines in increasing concentrations of methotrexate (1-10000 nM), new cell lines can be obtained which produce factor VIII protein at very high rate.

The above cell lines producing factor VIII protein can be grown on a large scale, either in suspension culture or on various solid supports. Examples of these supports are microcarriers based on dextran or collagen matrices, or solid supports in the form of hollow fibers or various ceramic materials. When grown in suspension culture or on microcarriers, the culture of the above cell lines can be performed either as a bath culture or as a perfusion culture with continuous production of conditioned medium over extended periods of time. Thus, according to the present invention, the above cell lines are well suited for the development of an industrial process for the production of recombinant factor VIII that can be isolated from human plasma.

The recombinant factor VIII proteins which accumulate in the medium of CHO-cells of the above type, can be concentrated and purified by a variety of biochemical methods, including, but not limited to, methods utilizing differences in size, charge, hydrophobicity, solubility, and/or specific affinity between the recombinant factor VIII protein and other substances in the cell cultivation medium.

An example of such a purification is the adsorption of the recombinant factor VIII protein to a monoclonal antibody which is immobilized on a solid support. After desorption, the factor VIII protein can be further purified by a variety of chromatographic techniques based on the above properties.

The recombinant proteins, with the activity of wild-type factor VIII, described in this invention can be formulated into pharmaceutical preparations for therapeutic use. The purified factor VIII proteins may be dissolved in conventional physiologically compatible aqueous buffer solutions to which there may be added, optionally, pharmaceutical adjuvants to provide pharmaceutical preparations.

In one embodiment, the present invention encompasses a method of treating hemophilia, comprising administering to a patient in which such treatment, prevention or amelioration is desired, a pharmaceutical preparation comprising a recombinant factor VIII protein of the invention in an amount effective to treat, prevent or ameliorate the disorder.

The modified factor VIII DNA's of this invention may also be integrated into a transfer vector for use in human gene therapy. Transfer vectors for use in human gene therapy include, but are not limited to, AAV, adenovirus, lentiviruses, HSV, and/or derivations thereof. Other vectors include, but are not limited to, those derived from viral sequences or sequences of nonviral origin that guarantee that a construct containing the FVIII cDNA and the sequences of nonviral origin, once it is introduced into a cell, are transported into the nucleus to allow for stable integration or at least stable propagation of the construct to ensure transcription of the FVIII cDNA and subsequent expression of FVIII by transfected cells.

The present invention will be further described more in detail in the following examples thereof.

### EXAMPLES

### Example 1

### Cloning of the Introns

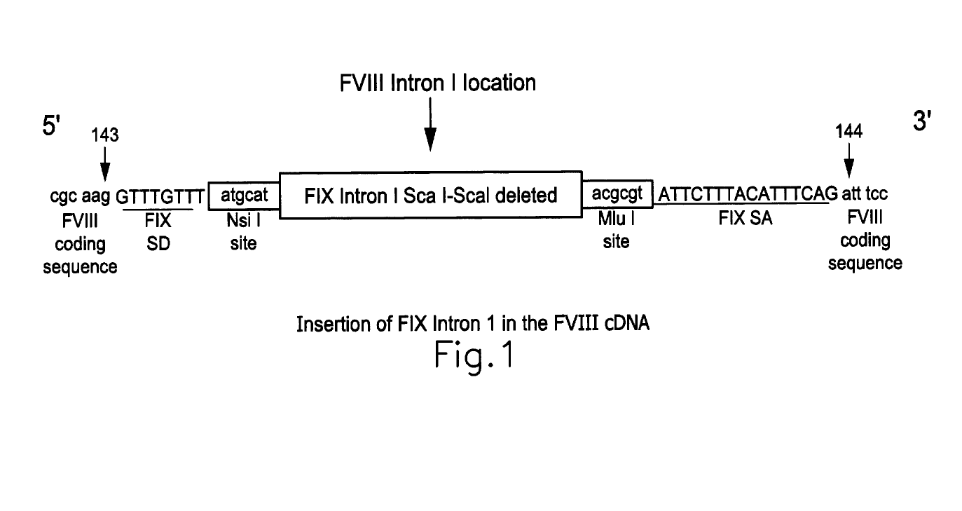

To clone the first introns of the apolipoprotein A1 (A) and the β-globin (B) genes in place of the FIX intron 1, two set of primers were designed. The new intronic sequences inserted between the splice donor (SD) and the splice acceptor (SA) of the FIX intron 1 are shown in FIG. 1. The two sets of oligonucleotides amplified the intron deleted of the respective SD and SA sites (5′ cloning site, Nsil and 3′ cloning site, Mlul). The following primers were used:

| Name | Intron target | sequence |
|---|---|---|
| APOAII-S | Apolipoprotein | CATGCATTGCTGCCTGCCCCGGTCAC |
| | Al (sense) | TC |
| | | (SEQ ID NO:1) |
| APOAII-AS | Apolipoprotein | TACGCGTCCTGGCTGAGTGGGGTGCC |
| | Al (antisense) | TT |
| | | (SEQ ID NO:2) |
| BGLOBI-S | R-Globin | CATGCATCAAGGTTACAAGACAGGTT |
| | (sense) | T |
| | | (SEQ ID NO:3) |
| BGLOBI-AS | S-Globin | TACGCGTGACCAATAGGCAGAGAGAG |
| | (antisense) | T |
| | | (SEQ ID NO:4) |

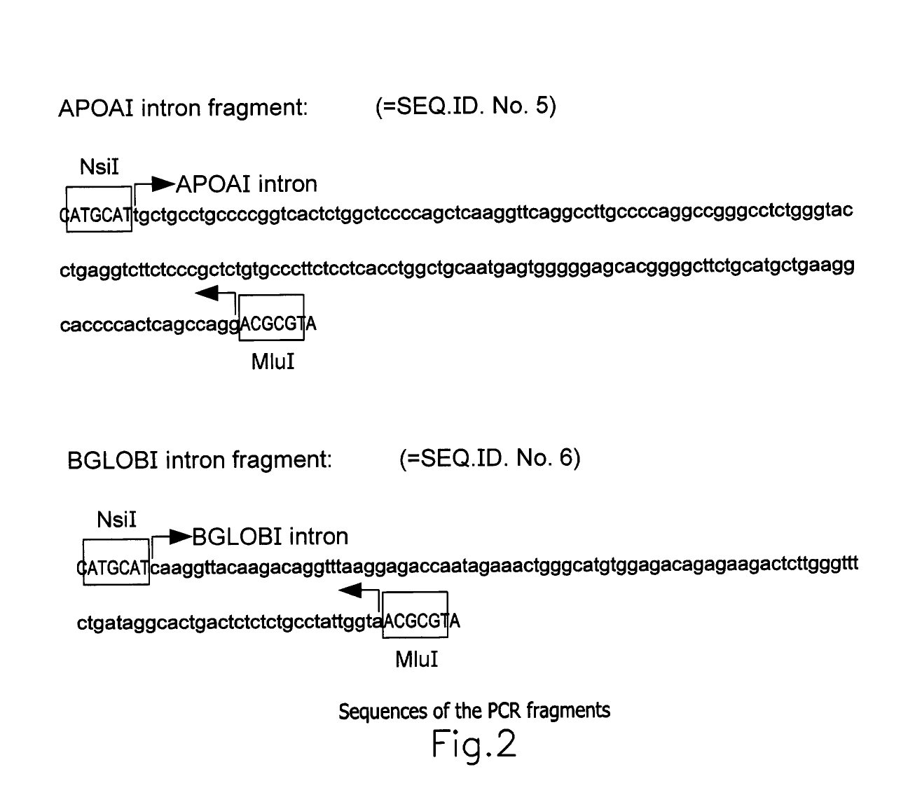

PCR reactions were performed using genomic DNA. The apolipoprotein AI intron I (APOAI intron) gave a 186 base pairs (bp) length fragment and the β-globulin intron I (BGLOBI intron) gave a 119 bp length fragment (FIG. 2). PCR fragments were cloned using TOPO TA cloning kit (pCR II vector: Invitrogen, Leek, the Netherlands).

### Example 2

### Insertion of Each Intron in Position 1 or 13 in the FVIII cDNA's

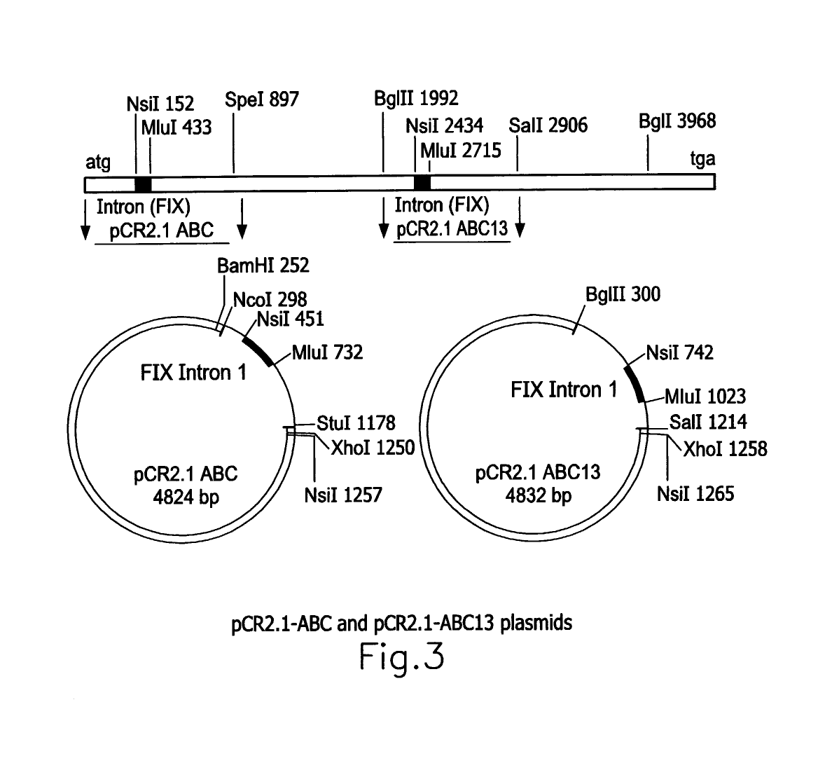

To insert the introns in the FVIII cDNA, two plasmids were used that were obtained during the initial cloning of the FIX intron 1 (FIG. 3). pCR2.1 ABC comprises the FVIII ATG fragment (Ncol-Spel) with the FIX intron in position 1. pCR2.1 ABC13 contains a Bglil-Sall fragment of the FVIII cDNA with the FIX intron in position 13.

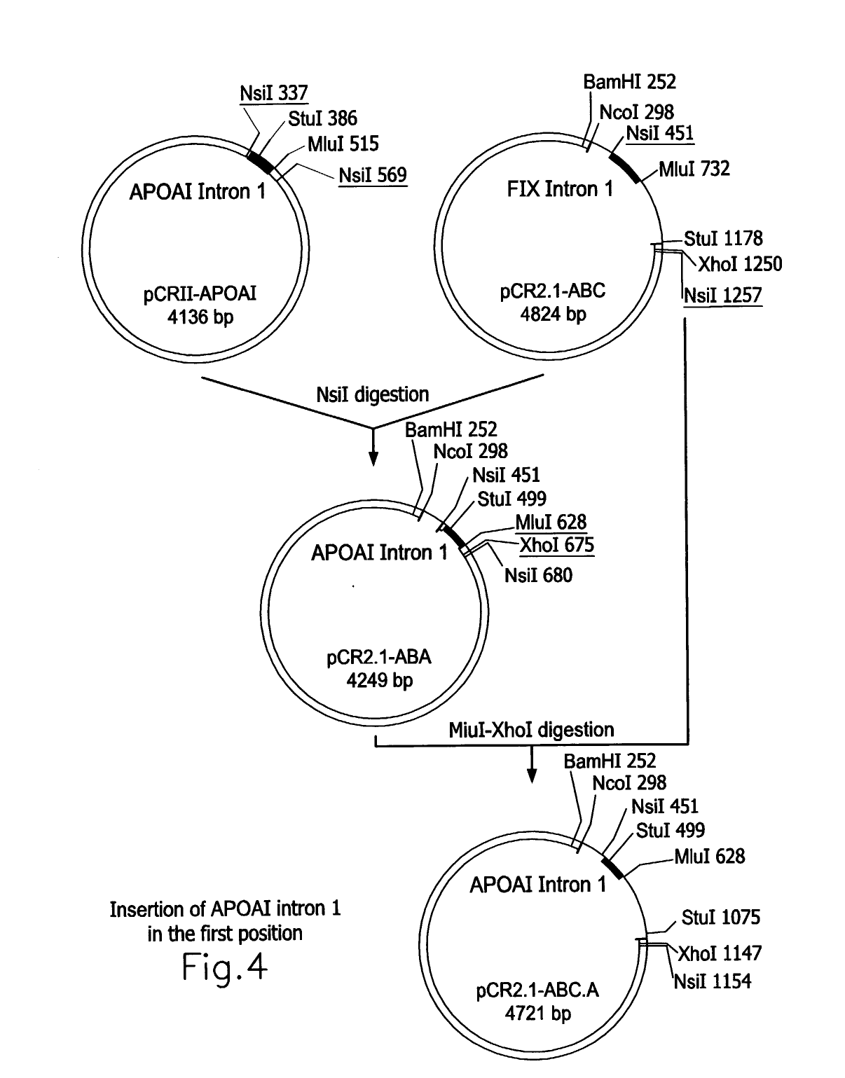

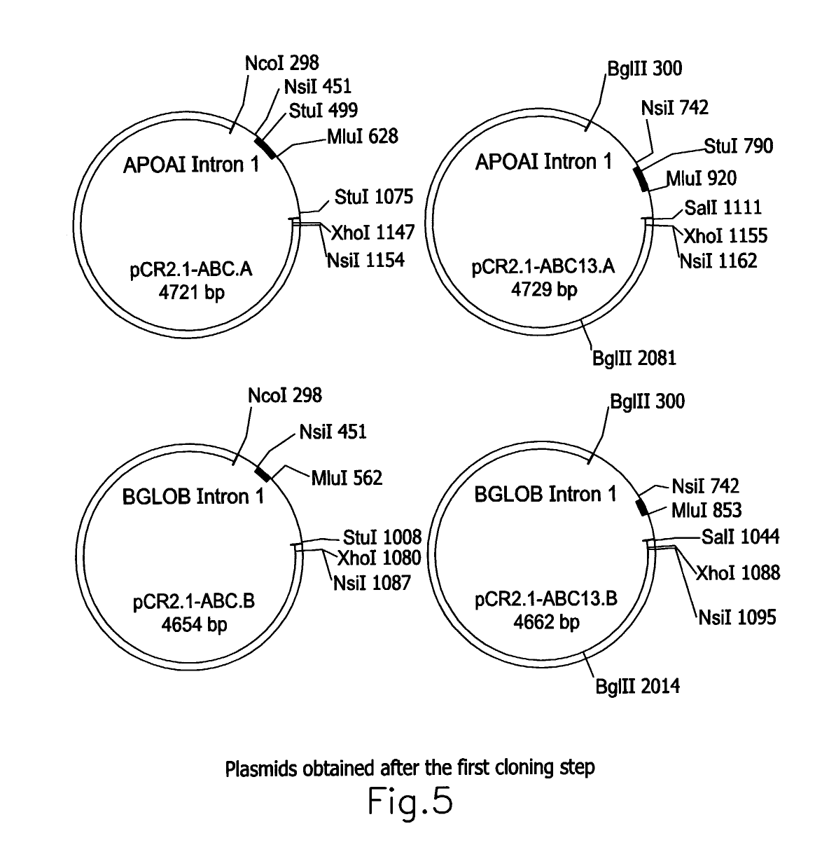

The APOAI intron was inserted in the pCR2.1 ABC using a Nsil digestion (FIG. 4). The Mlul-Xhol fragment of the pCR2.1 ABC was thereafter re-introduced in the obtained vector. The final plasmid was pCR2.1 ABC.A, comprising the ATG fragment of the FVIII with APOAI intron in position 1. The same strategy was used to clone the BGLOB intron in position 1. The same Nsil ligation, followed by the re-introduction of the Mlul-Xhol fragment, was also used for insertion into position 13. The obtained vectors are presented in FIG. 5.

### Example 3

### Construction of the FVIII A1+13 and FVIII B1+13 constructs

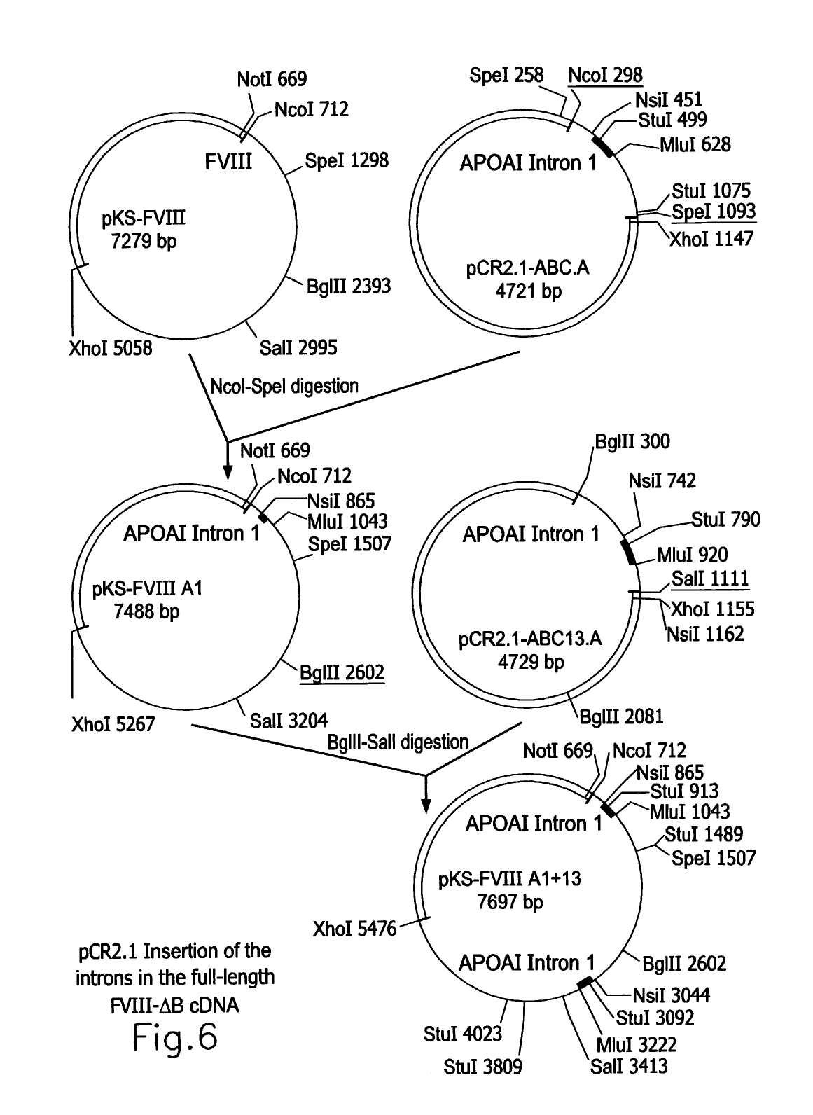

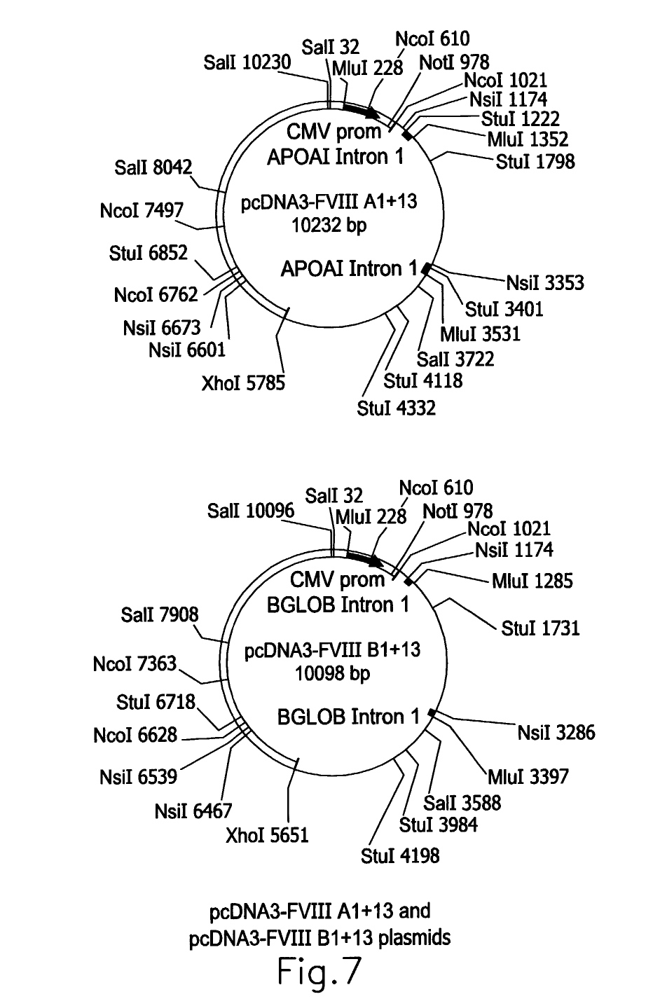

pKS-FVIII contains the B domain-deleted FVIII cDNA. The plasmid was opened with Ncol and Spel enzymes (FIG. 6), as was pCR2.1 ABC.A. The insert originating from pCR2.1 ABCA was inserted into pKS-FVIII, opened by Ncol and Spel. After ligation, PKS-FVIII AI was obtained. This vector comprised the APOAI intron in position 1 and was subsequently digested with BgIII-SaII digestion in order to introduce the intron in position 13. The final plasmid was pKS-FVIII A1+13 exhibiting the 2 intronic sequences. The same strategy was used to obtain pKS-FVIII B1+13. These two plasmids were subsequently digested by NotI and XhoI and the inserts were ligated in pcDNA3 vector (Invitrogen, Leek, the Netherlands), opened by the same enzymes. The final expression plasmids were called pcDNA3-FVIII AI+13 and pcDNA3 FVIII B1+13 (FIG. 7).

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A modified factor VIII CDNA, comprising at least one spliceable nucleotide sequence that is inserted into the wild-type factor VIII cDNA at the original position of at least one intron of the genomic FVIII DNA, wherein said at least one intron is selected from the group consisting of intron 1 and intron 13.

2. Modified factor VIII cDNA as specified in claim 1, wherein said at least one spliceable nucleotide sequence is a synthetic intron or a fragment thereof.

3. Modified factor VIII cDNA as specified in claim 2, wherein said synthetic intron is selected from the group consisting of:
(a) Apolipoprotein I intron 1; and
(b) β-Globulin intron 1.

4. A recombinant expression vector comprising the modified factor VIII cDNA as specified in claim 1, operably associated with a transcriptional promoter and a polyadenylation sequence.

5. A host cell of animal origin comprising the recombinant vector of claim 4.

6. A method for producing a protein, comprising:
(a) culturing the host cell of claim 5 under conditions suitable to produce a polypeptide encoded by the modified human factor VIII cDNA of claim 1; and
(b) recovering said polypeptide from the cell culture medium.

7. A protein produced by the method of claim 6.

8. A composition comprising the protein as specified in claim 7 and a pharmaceutically acceptable carrier.

9. A method for treating hemophilia comprising administering to a human the pharmaceutical composition of claim 8.

10. A transfer vector for use in human gene therapy, comprising the modified factor VIII cDNA as specified in claim 1.

11. A modified factor VIII cDNA, comprising:
(a) a first DNA segment coding for amino acids 1 through 740 of the human factor VIII protein;
(b) a second DNA segment coding for amino acids 1649 through 2332 of the human factor VIII protein;
(c) a linker DNA segment encoding at least two amino acids, connecting said first DNA segment and said second DNA segment, wherein said amino acids are selected from the group consisting of lysine and arginine; and
(d) at least one spliceable nucleotide sequence that is inserted into the original position of at least one intron of the genomic FVIII DNA, wherein said at least one intron is selected from the group consisting of intron 1 and intron 13.

12. Modified factor VIII cDNA as specified in claim 11, wherein said spliceable nucleotide sequence is a synthetic intron or a fragment thereof.

13. Modified factor VIII cDNA as specified in claim 12, wherein said synthetic intron is selected from the group consisting of:
(a) Apolipoprotein I intron 1; and
(b) β-Globulin intron 1.

14. A recombinant expression vector comprising the modified factor VIII cDNA as specified in claim 11, operably associated with a transcriptional promoter and a polyadenylation sequence.

15. A host cell of animal origin comprising the recombinant vector of claim 14.

16. A method for producing a protein, comprising:
(a) culturing the host cell of claim 15 under conditions suitable to produce a polypeptide encoded by the modified human factor VIII cDNA of claim 1; and
(b) recovering said polypeptide from the cell culture medium.

17. A protein produced by the method of claim 16.

18. A composition comprising the protein as specified in claim 17 and a pharmaceutically acceptable carrier.

19. A method for treating hemophilia comprising administering to a human the pharmaceutical composition of claim 18.

20. A transfer vector for use in human gene therapy, comprising the modified factor VIII cDNA as specified in claim 11.


SEQUENCE LISTING (6 sequences)

SEQ ID NO: 1 - DNA, 28 bp, Artificial Sequence
  5′ APOAI intron primer with NsiI restriction site
catgcattgctgcctgccccggtcactc

SEQ ID NO: 2 - DNA, 28 bp, Artificial Sequence
  3′ APOAI intron primer with MluI restriction site
tacgcgtcctggctgagtggggtgcctt

SEQ ID NO: 3 - DNA, 27 bp, Artificial Sequence
  5′ BGLOBI intron primer with NsiI restriction site
catgcatcaaggttacaagacaggttt

SEQ ID NO: 4 - DNA, 27 bp, Artificial Sequence
  3′ BGLOBI intron primer with MluI restriction site
tacgcgtgaccaataggcagagagagt

SEQ ID NO: 5 - DNA, 186 bp, Homo sapiens
catgcattgctgcctgccccggtcactctggctccccagctcaaggttcaggccttgccccaggccgggcctctgggtacctgaggtcttctcccgctctgtgcccttctcctcacctggctgcaatgagtgggggagcacggggcttctgcatgctgaaggcaccccactcagccaggacgcgta

SEQ ID NO: 6 - DNA, 119 bp, Homo sapiens
catgcatcaaggttacaagacaggtttaaggagaccaatagaaactgggcatgtggagacagagaagactcttgggtttctgataggcactgactctctctgcctattggtcacgcgta